(19) United States
(12) Patent Application Publication
(10) Pub. No.: US 2007/0112337 A1
(43) Pub. Date: May 17, 2007

(54) Endoscope with Protective Sleeve

(75) Inventors: Golan Salman, Atlit (IL); Niv Sher, Kiriat Motzkin (IL)

Correspondence Address: BRUCE E. LILLING;LILLING & LILLING PLLC, P.O. BOX 560, GOLDEN BRIDGE, NY, 10526, US

(73) Assignee: STRYKER GI LTD., Haifa (IL)

(21) Appl. No.: 11/560,482

(22) Filed: Nov. 16, 2006

Related U.S. Application Data
(60) Provisional application No. 60/738,001, filed on Nov. 17, 2005.

(51) Int. Cl. A61B 17/00

(52) U.S. Cl. 606/1

(57) ABSTRACT

A protective sleeve is disclosed for protecting a flexible shaft of an endoscopic tool from contamination or the environment. The protective sleeve comprises a first protective sleeve portion for receiving the shaft of the endoscopic tool, a second protective portion, which is attachable to a port of an endoscope, and a tubular guide member through which the shaft of the endoscopic tool can be longitudinally displaced when the shaft is being either retracted from a port of an endoscope or protracted thereinto.

### BACKGROUND OF THE INVENTION

1. Field of the Invention

The present invention relates generally to endoscopy, and specifically to a protective sleeve for sheathing an endoscopic tool after its withdrawal from a body passage.

2. Summary of the Invention

The use of a disposable sleeve (also referred to as a sheath) to cover an endoscope is well known in the art. Flexible endoscopes, such as colonoscopes, are notoriously difficult to clean and disinfect thoroughly, leading to problems of cross-contamination between patients and between patients and staff. These problems can be avoided by covering the endoscope with a single-use sleeve, which is discarded after use.

Endoscopes commonly have working channels, running from a proximal port outside the body to a distal port at the distal end of the endoscope. When the distal end of the endoscope is inserted into the body, the working channel may be used, inter alia, to pass a surgical instrument through to the distal end of the endoscope in order to perform a surgical procedure, such as a biopsy. Instruments that are used in this manner become contaminated with biological matter from inside the patient's body. As the instrument is withdrawn from the body, it spreads the contamination to the interior of the working channel and to the proximal port of the endoscope and to the operator's hands.

Silverstein (U.S. Pat. No. 5,695,491) discloses a containment system for containment of at least a major portion of the shaft of the working tool. However, this containment system is adapted to contain the shaft remote from the endoscope and it is not designed to cover that portion of the shaft, which is between the containment container and the endoscope.

Methods for sheathing an endoscope while providing working channels that protect the endoscope from contamination are described, for example, in Silverstein (U.S. Pat. No. 4,646,722) and Sidall (U.S. Pat. No. 4,741,326), whose disclosures are incorporated herein by reference. These patents attempt to prevent contamination of the endoscope, either by adding disposable working channels external to the endoscope itself (Silverstein—U.S. Pat. No. 4,646,722) or by adding a disposable liner inside a channel of the endoscope (Sidall—U.S. Pat. No. 4,741,326). They do not address the problem, however, of contamination that may be spread to the area around the proximal end of the endoscope and to the operator's hands as the surgical tool is retracted from the proximal port of the working channel.

An attempt to solve this problem is described for example in Aizenfeld (U.S. Pat. No. 6,908,428), which is incorporated herein by reference. This patent discloses methods and devices for use in sheathing an endoscopic tool as it is removed from the patient's body. Such sheathing prevents contaminants that may adhere to the tool inside the body or from the contaminated working channel from contacting the operator's hands, the handle of the endoscope, or other objects outside the patient's body. As a result, the likelihood of cross-contamination between patients is reduced, and the job of cleaning and disinfecting the endoscope and ancillary equipment between uses is simplified.

In embodiments of this solution, a sheathing assembly is provided, which comprises a sleeve dispenser mating with the proximal port of an endoscopic working channel, outside the patient's body. A flexible sleeve is typically fixed by its distal end to the dispenser, with the remainder of the sleeve bunched inside or otherwise held in a vicinity of the dispenser. An elongate endoscopic tool is passed through the dispenser and the working channel, until the distal end of the tool protrudes from the distal end of the endoscope. While the shaft of the tool is advanced through the dispenser and the proximal port of the working channel, the sleeve remains bunched at the dispenser. When the tool is retracted, however, the proximal end of the sleeve engages the shaft of the tool, so that as the tool is withdrawn, the sleeve unfurls from the dispenser to cover the shaft of the tool, up to and including its distal end. All contaminants on the tool thus remain within the sleeve, while the outside of the sleeve remains clean and can be handled freely without spreading contamination.

Unfortunately this solution suffers from the fact that when the tool is being retracted from the working channel, there exists a possibility that the distal end of the sleeve, which is made of non-elastometic material, might be inadvertently torn and detached from the location where it is anchored to the sheathing assembly. Accordingly the tool might become exposed and contaminations adhered to the tool would spread to the environment.

Another disadvantage of the above solution lies in the fact that after the tool has been already retracted from the working channel and is protracted therein again, the sleeve bunches immediate before the sheathing assembly and impedes the doctor's fingers to grasp the tool as close as possible to the proximal port as would be desirable for easy and sure advancement of the tool inside the port.

Moreover, since the tool shaft deflects within the bunched portion, this opposes the advancement of the tool and renders it difficult.

The present invention seeks to eliminate the above-mentioned disadvantages of the known solution.

### BRIEF DESCRIPTION OF THE SEVERAL VIEWS OF THE DRAWINGS

The present invention will be more fully understood from the following detailed description of the embodiments thereof, taken together with the drawings in which:

FIG. 1 is a schematic, pictorial illustration of a system for performing an endoscopic procedure, in accordance with an embodiment of the present invention;

FIG. 2 is a schematic isometric view of protective sleeve of the present invention without the endoscopic tool;

FIG. 3 is a broken away view of the protective sleeve shown in FIG. 2, showing the interior without the endoscopic tool;

FIG. 4 is a view similar to FIG. 3, but depicting the protective sleeve assembly of the invention with the endoscopic tool inserted in the assembly and slightly protruding from the assembly into the proximal port of the endoscope;

FIG. 5 is a view similar to FIGS. 3 and 4, but depicting the protective sleeve assembly of the invention with the endoscopic tool fully inserted in the assembly and extending along the endoscope;

FIG. 6 is a view, showing how the tubular guide member of the protective sleeve assembly is detached from the distal fitting and the protective sleeve is about to extend from the bunched condition to the extended condition;

FIG. 7 schematically shows displacement of the endoscopic tool forward through the protecting sleeve assembly; and

FIG. 8 schematically shows displacement of the endoscopic tool backward through the protecting sleeve assembly.

### DETAILED DESCRIPTION OF THE PREFERRED EMBODIMENT

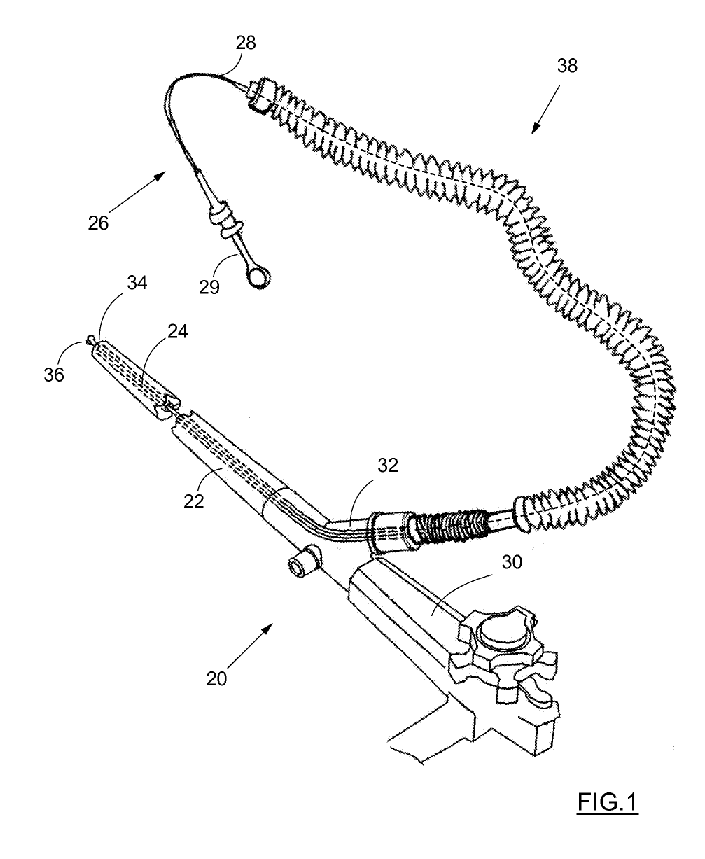

FIG. 1 is a schematic, pictorial illustration of an endoscopic system 20 for performing an endoscopic procedure, in accordance with an embodiment of the present invention.

System 20 comprises an endoscope 22, having a working channel 24 passing therethrough. Channel 24 passes through endoscope 22 from a proximal port 32, typically in or near an operating handle 30 of the endoscope, to a distal port 34 at the distal end of the endoscope. An endoscopic tool 26 is inserted through working channel 24 in order to access an area adjacent to the distal end of the endoscope, within the patient's body. Typically, endoscopic tool 26 comprises an elongate shaft 28, with a working element 36 at its distal end and with a working handle 29 at its proximal end, as is known in the art. In the example shown in FIG. 1, working element 36 comprises biopsy forceps, which are operable to take a tissue sample within the patient's body, adjacent to distal port 34. Alternatively or additionally, channel 24 may be used to apply suction to a body passage through distal port 34 or to apply fluid or gas to the area outside the distal port, as is likewise known in the art.

A disposable sheath may cover insertion tube of endoscope 22, and channel 24 may likewise be internally sheathed, in order to protect the endoscope from contamination, e.g. as described in Sidall (U.S. Pat. No. 4,741,326). Alternatively or additionally, although channel 24 is shown in the figure as passing inside the endoscope, the endoscopic working channel may comprise a separate tube, typically disposable, which is retained alongside the endoscope, as described, for example, in Silversstein (U.S. Pat. No. 4,646,722). The present invention is suited for use with either of these types of working channels.

Since at least working element 36 of tool 26 comes into contact with tissue and other biological matter inside the patient's body, the tool and the interior of the working channel (or the internal sheath which provides lining for the working channel) necessarily become contaminated during use. In order to prevent the spread of contamination from tool 26 to handle 30, to the operator's hands and to other areas outside the body, a protective sleeve 38 is attached to port 32. Protective sleeve 38 is connected to port 32 in such a manner that a passage through the sleeve is aligned with working channel 24.

In FIG. 1 the protective sleeve is shown in a working state, i.e. in a situation when tool 26 has been inserted into working channel 24 (nearly to its full length) and its shaft is covered by the protective sleeve and the tool can be used in the usual manner.

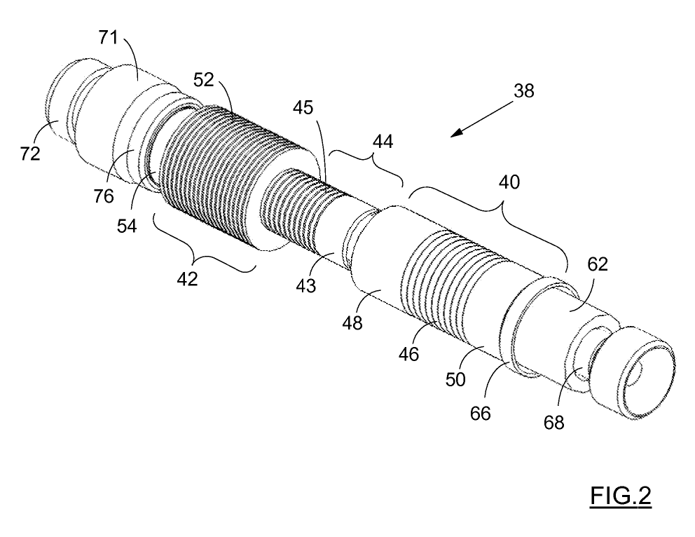

FIG. 2 is a schematic isometric view of protective sleeve 38, in accordance with an embodiment of the present invention. The protective sleeve is shown in an initial compacted state, when it is separate from the tool while being ready to receive it. The protective sleeve comprises its main components, which are a first protective sleeve portion 40 and a second protective sleeve portion 42 which will be referred-to also as an elastomeric sleeve portion. The opposite ends 43, 45 of the first protective sleeve portion and of the elastomeric sleeve portion overlap along a bridging region 44 where they are connected therebetween, e.g. by gluing. A major portion 46 of the first protective sleeve portion is bunched, while its distal 48 and proximal 50 extremities are not. A major portion 52 of the elastomeric sleeve portion is also bunched, while its distal extremity 54 and its proximal end 45 are not bunched. In accordance with the invention the first protective sleeve portion and the elastomeric sleeve portion are both compactly bunched during an initial state, i.e. before the tool is entered in the protective cover. During the working state the first protective sleeve portion unfurls to sheath the instrument shaft, while the elastomeric sleeve portion is elastically stretched.

The major portions of the first protective sleeve portion and the elastomeric sleeve portion have approximately the same diameter, while their diameter along the bridging region is less. The first protective sleeve portion is made of a thin polymeric material, e.g. Nylone, and has a thickness of about 10-30 micron. In practice it is advantageous if the first protective sleeve portion is made of a transparent material. The material of the first protective sleeve portion is selected such, that upon pulling its one end, it unfurls without however being stretched.

In order to bring it in the initial bunched state the first protective sleeve portion should be forcibly pushed in the distal direction. The second sleeve portion, or elastomeric sleeve portion, has a thickness of 0.1-0.5 mm. In contrast to the first protective sleeve portion the elasytomeric sleeve portion is made of a polymeric material, which is elastically stretchable upon pulling one of the sleeve ends. Therefore it is capable of returning by itself to the initial bunched state upon release. An example of suitable materials for the elastomeric sleeve portion are Silicone, Polyurethane, etc.

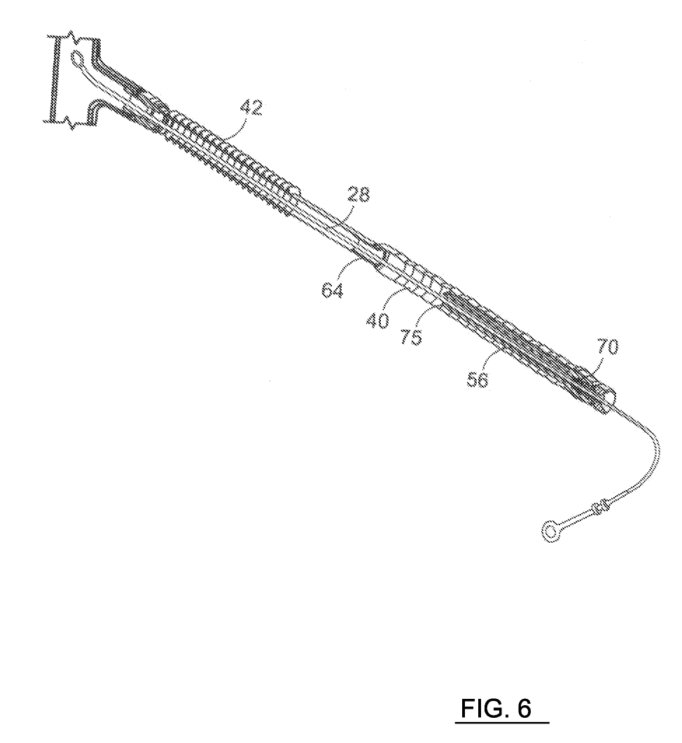

It will be explained further with reference to FIGS. 6-8, that in the working state the unfurling of the first protective sleeve portion is associated with retracting the tool from the endoscope, while bunching the first protective sleeve portion back is associated with protracting the tool into the endoscope.

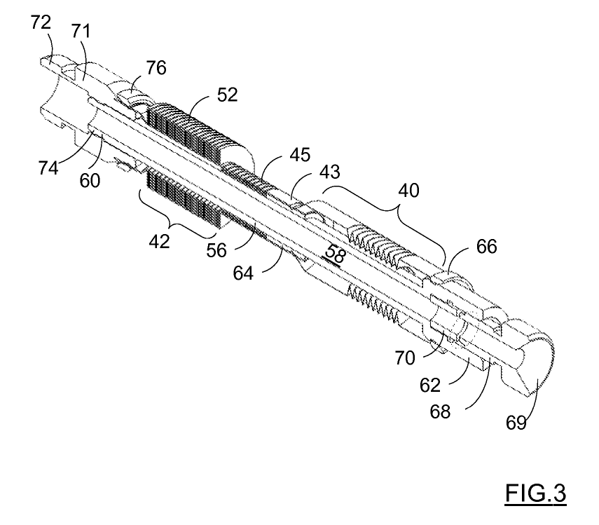

Now with reference to FIG. 2 and FIG. 3 still further components of the protective sleeve will be explained. Extending along the protective sleeve a tubular guide member 56 is provided, around which the first protective sleeve portion 40 and the elastomeric sleeve portion 42 are deployed with possibility for their longitudinal displacement with respect to the guide member. The guide member has a through going bore 58 through which the endoscopic tool passes when it is being displaced along the protective sleeve.

The guide member is made of a rigid plastic material, e.g. ABS, PVC, etc. The guide member extends longitudinally along the sleeve and has a distal end 60, a proximal end 62 and an intermediate tubular portion. The elastomeric sleeve portion is deployed close to distal end 60 of the guide member, while the first covering sleeve portion is deployed close to proximal end 62 of the guide member. An adapter bushing 64 is provided, which is put over the intermediate portion of the guide member with possibility for relative longitudinal displacement therebetween. The respective ends of both sleeve portions overlap at the bridging region 44 and are anchored to the adapter bushing by a suitable means, e.g. by an elastic ring or by gluing.

A snap ring 66 is provided for attaching non-bunched proximal extremity 50 of the first protective sleeve portion to an outside periphery of proximal end 62 of the guide member. Proximal end 62 of the guide member has an entry opening for receiving a hollow securing nut 68, which is screwable into the opening. Situated within proximal end 62 and located before the entry opening an expansion bushing 70 is provided. This bushing is made of resilient material and therefore is transversally expandable upon screwing the nut. By virtue of this provision it is possible to secure proximal end of the endoscopic tool at the proximal end of the guide member when the tool is being inserted into the sleeve through a flared end 69 of the nut and further through the nut and the bore of the guide member. It should be borne in mind, however, that alternative methods for securing the proximal end of the tool shaft can be used, e.g. it can be a plug, which is made of elastic material and contains an aperture that fits snugly around the shaft and enables advancement thereof through the plug only in the distal direction.

Releasable attached to the distal end of the guide member, a distal fitting 71 is provided, having a nipple portion 72 for connecting to proximal port 32 of the endoscope.

In practice releasable attachment can be achieved by a snap connection, which would comprise annular protrusion 74 made on the distal end of the guide member and correspondingly mating annular groove made on an inside periphery of the distal fitting. By virtue of this provision the guide member can be easily detached from the fitting.

A snap ring 76 is provided for anchoring distal extremity 54 of the elastomeric sleeve portion to an outside periphery of the distal fitting. Optionally the elastomeric sleeve portion could be connected to the fitting by gluing.

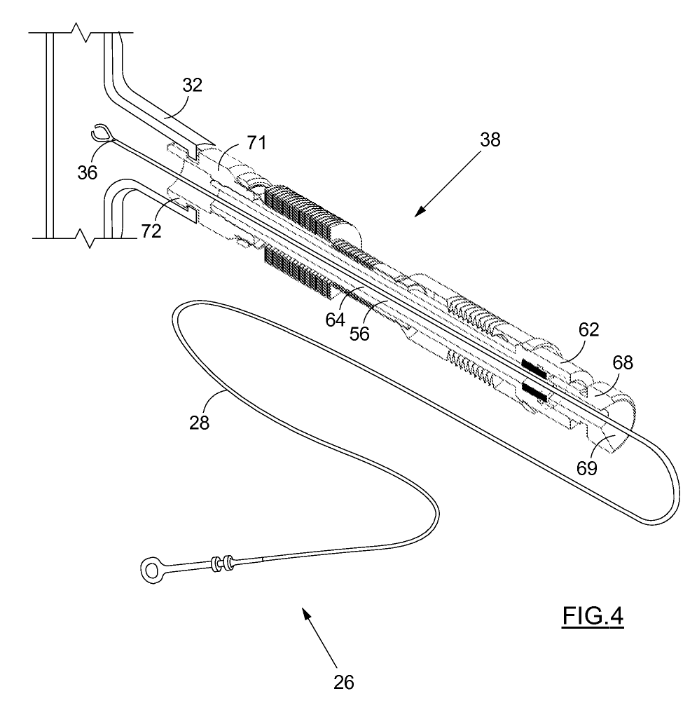

Attention is called now to FIG. 4, in which the protective sleeve of the invention is depicted in the beginning of the working state. In this situation distal fitting 71 is attached to proximal port 32 of the endoscope and then endosocpic tool 26 has been inserted into first protective sleeve portion 40 through flaring end 69 of securing nut 68. In practice the order in which the protective sleeve is brought in this state can be different. It is possible either to start from inserting the tool in the first protective sleeve portion and then to attach the fitting to the endoscope port or alternatively to attach the fitting first and then to insert the tool.

FIG. 4 shows that the tool has been advanced in the distal direction until its working element 36 passes guide member 64 and then slightly protrudes from the guide member. Since the nut is not fully screwed in the proximal end 62 of the guide member, the tool shaft is free to advance in the distal direction.

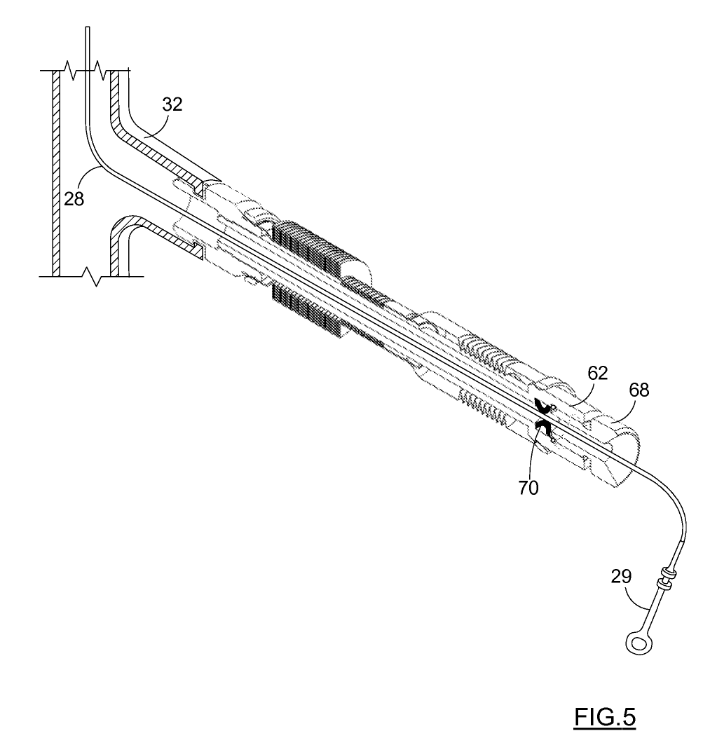

In FIG. 5 is shown still further step when the tool has been fully advanced in the port so that its shaft 28 is almost entirely within the endoscope and handle 29 is brought close to the proximal end of the first protective sleeve portion. In this position the endoscopic tool can be used for taking a biopsy sample. The securing nut 68 is screwed in the opening of the proximal end such that it deforms bushing 70 laterally and it fixes proximal end of the tool shaft at the proximal end of the first protective sleeve portion.

Referring to FIG. 6 it is shown the situation when the tool is in the beginning of its retraction back from the proximal port. It is seen that guide member 56 is pulled backwards in the proximal direction and its distal end 75 is detached from the connection fitting. The proximal end of tool shaft 28 is secured at the distal end of the guide member and thus the guide member and the tool shaft retract simultaneously in the proximal direction.

At the same time the first protective sleeve portion 40, which has one end secured on the bushing 64 and the opposite end secured to the proximal end of the guide member, continues to unfold and cover the tool shaft.

Eventually the tool shaft is fully retracted from the port and upon detachment of the fitting from the port, the biopsy sample can be transferred into a dedicated sample container.

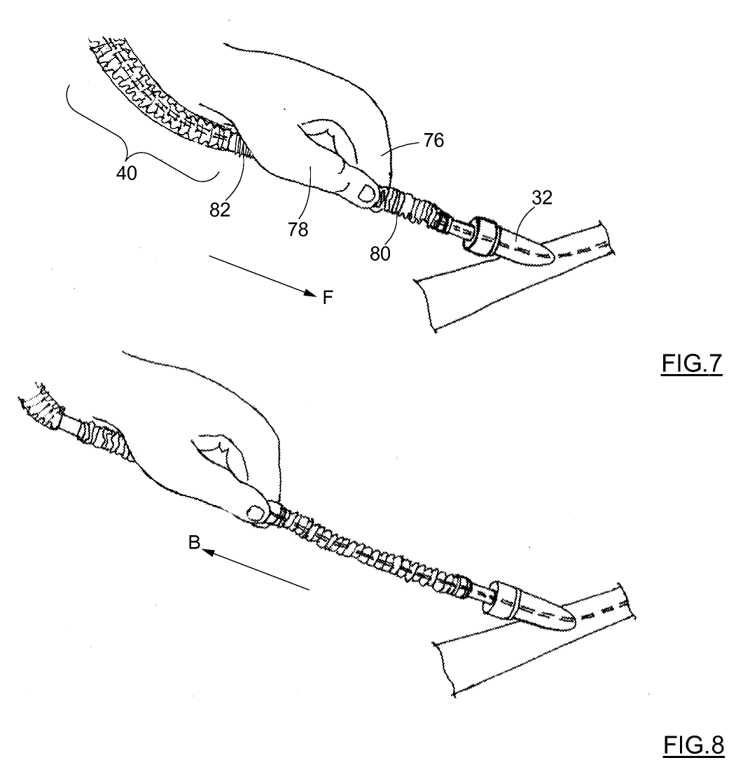

For further protracting or retracting of the tool one should grasp the tool shaft through the elastomeric sleeve portion by fingers in an initial position close to the port 32, as shown in FIGS. 7 and 8, and then to displace the shaft forward or backward while squeezing the elastomeric sleeve portion. In FIGS. 7,8 is shown how doctor's fingers 76, 78 squeeze the elastomeric sleeve portion while grasping the tool shaft. Then the shaft is displaced in forward or backward direction as designated by respective arrows F,B. The tool shaft is displaced by virtue of a “milking” movement, which comprises repetitive advancing of the shaft forward or backward from the initial position, then release of the shaft in a new position, while still grasping the elastomeric sleeve, and then returning the fingers to the initial position.

In the prior art endoscopes, which do not employ protecting sleeves, the doctors are accustomed to protracting the tool in the port and retracting it from the port by the same way and therefore they should not change their habit while using the protective sleeve of the present invention.

It can be readily appreciated that, when the doctor's fingers advance the tool shaft forward to protract it in the port, the elastomeric sleeve portion slightly bunches between the initial position and the fitting. This situation is depicted in FIG. 7, in which the bunched region of the elastomeric sleeve portion is designated by reference numeral 80. The portion of the first protective sleeve portion behind the fingers is designated by reference numeral 82. When the elastomeric sleeve portion bunches the first protective sleeve portion is pulled in the distal direction and straightens behind the fingers.

At the same time when the shaft is displaced backward, i.e. is retracted from the port, as seen in FIG. 8, the elastomeric sleeve portion is resiliently stretched and causes the first protective sleeve portion to bunch and gather behind the fingers. It should be borne in mind, however, that when either the elastomeric portion or the first protective sleeve portion bunches, it nevertheless does not provide a hindrance for protracting or retracting the tool shaft and the doctor's fingers can always remain close to the proximal port of the working channel. By virtue of this provision advancement of the tool shaft through the port is easy, reliable and efficient.

Furthermore, since the elastically stretchable elastomeric sleeve portion would act as a spring, the probability for its detachment from fitting 71 is much less.

In practice the covering sleeve of the invention is used as follows. First, the preparation step is carried out, during which the tool shaft is entered into the proximal end of the guiding member and then pushed forwards until the working element 36 of the tool slightly protrudes from the fitting. After that, connection fitting is attached to proximal port 32 of the endoscope and the tool is protracted thereinto until the tool's operating handle is in vicinity to the proximal end of the guiding member as shown in FIG. 5. In this position the rear portion of the tool shaft can be secured at the proximal end of the guiding member by securing nut 68.

After completing the preparation step the tool shaft can be either retracted from proximal port 32 or protracted thereinto by virtue of the above described “milking” movement applied to the elastomeric sleeve portion.

Up to now an embodiment of the protective sleeve has been described, in which the cover constitutes a sole item, which is independent from the endoscopic tool. This item can be supplied separately from the tool and for its use during the endoscopic procedure one should complete the above described preparation step.

However, one can contemplate also a situation, in which the cover and the tool are supplied as a unitary item, i.e. as a protective cover assembly in which the tool has been already inserted in the guiding member, its shaft is secured at the proximal end of the guiding member and the working tool is ready for protracting into the colonoscope upon connection of the fitting 71 with proximal port 32.

It will thus be appreciated that the embodiments described above are cited by way of example, and that the present invention is not limited to what has been particularly shown and described hereinabove. Rather, the scope of the present invention includes both combinations and sub combinations of the various features described hereinabove, as well as variations and modifications thereof which would occur to persons skilled in the art upon reading the foregoing description and which are not disclosed in the prior art.

We claim:

1. A protective sleeve for protecting a flexible shaft of an endoscopic tool, said protective sleeve comprising
a first protective sleeve portion for receiving the shaft of the endoscopic tool, said first protective sleeve portion being configured as a sleeve having an intermediate region, a proximal end through which the shaft of the endoscopic tool can be protracted within the first protective sleeve portion, and a distal end, said intermediate region being bunched, and wherein upon pulling of one end said first protective sleeve portion unfuring without being elastically stretched,
a second protective portion having an intermediate bunched region, a proximal end and a distal end, said distal end being attachable to a port of an endoscope, wherein said intermediate bunched region being elastically stretchable upon pulling of one of its ends, and
a tubular guide member through which the shaft of the endoscopic tool being longitudinally displaced when the shaft of the endoscopic tool is being either retracted from a port of an endoscope or protracted thereinto,
wherein the distal end of the first protective portion and the proximal end of the second protective portion being connected to the tubular guide member so as to bring the first protective sleeve portion in flow communication with the second protective sleeve portion, wherein bunching of the second protective sleeve portion being associated with unfurling of the first protective sleeve portion and with retracting the shaft of the endoscopic tool from the port, and wherein elastic stretching of the second protective sleeve being associated with bunching of the first protective sleeve portion and with protracting the shaft of the endoscopic tool into the port.

2. The protective sleeve as defined in claim 1, wherein said first protective sleeve portion being made of a thin polymeric material.

3. The protective sleeve as defined in claim 1, wherein said first protective sleeve portion being made of Nylon.

4. The protective sleeve as defined in claim 1, wherein said second protective sleeve portion being made of an elastomeric material.

5. The protective sleeve as defined in claim 4, wherein said elastomeric materials being selected from the group consisting of Silicone rubber and Polyurethane.

6. The protective sleeve as defined in claim 1, wherein said guide member being made of a rigid plastic material.

7. The protective sleeve as defined in claim 6, wherein said plastic material being selected from the group consisting of ABS and PVC.

8. The protective sleeve as defined in claim 1, wherein the proximal end of the first protective sleeve portion being provided with a securing means to allow a rear part of the shaft of the endoscopic tool to be only protracted through the proximal end and retraction through the proximal end being prevented.

9. The protective sleeve as defined in claim 8, wherein said securing means being a one-way plug.

10. The protective sleeve as defined in claim 1, wherein the distal end of said second protective sleeve portion being provided with an adapter for connecting to the port of the endoscope.

11. The protective sleeve as defined in claim 1, wherein said distal end of the first protective sleeve portion overlaps with the proximal end of the second protective sleeve portion.

12. A protective sleeve assembly comprising in combination
an endoscopic tool provided with a flexible shaft and
a protective sleeve for protecting at least the flexible shaft of the endoscopic tool, wherein said protective sleeve comprises
a first protective sleeve portion for receiving the flexible shaft of the endoscopic tool, said first protective sleeve portion being configured as a sleeve having an intermediate region, a proximal end through which the flexible shaft can be protracted within the first protective sleeve portion, and a distal end, said intermediate region being bunched, and wherein upon pulling of one end said first protective sleeve portion unfurling without being elastically stretched,
a second protective portion having an intermediate bunched region, a proximal end and a distal end attachable to a port of an endoscope, wherein said intermediate bunched region being elastically stretchable upon pulling of one of its ends,
a tubular guide member through which the flexible shaft of the endoscopic tool can be longitudinally displaced when the flexible shaft is being either retracted from a port of an endoscope or protracted thereinto,
wherein the distal end of the first protective portion and the proximal end of the second protective portion being connected to the tubular guide member to bring the first protective sleeve portion in flow communication with the second protective sleeve portion, wherein bunching of the second protective sleeve portion bring associated with unfurling of the first protective sleeve portion and with retracting the flexible shaft from the port, and wherein elastic stretching of the second protective sleeve being associated with bunching of the first protective sleeve portion and with protracting the flexible shaft into the port of the endoscope.

13. The protective sleeve assembly as defined in claim 12, wherein the proximal end of said first protective sleeve portion being provided with a securing means to allow a rear part of the flexible shaft to be only protracted through the proximal end and retraction through the proximal end being prevented.

14. The protective sleeve assembly as defined in claim 12, wherein the distal end of said second protective sleeve portion being provided with an adapter for connecting to the port of the endoscope.

15. An endoscopic system comprising
an endoscope fitted with a working channel passing therethrough from a proximal port situated near an operating handle to a distal port;
an endoscopic tool insertable through the working channel in order to access an area adjacent to the distal port of the endoscope, said endoscopic tool having a flexible shaft and a working element at a distal end thereof;
a protective sleeve for protecting a flexible shaft of an endoscopic tool, said protective sleeve comprising
a first protective sleeve portion for receiving the shaft of the endoscopic tool, said first protective sleeve portion being configured as a sleeve having an intermediate region, a proximal end through which the tool shaft can be protracted within the first protective sleeve portion, and a distal end, said intermediate region being bunched, and wherein upon pulling of one end said first protective sleeve portion unfurling without being elastically stretched,
a second protective portion having an intermediate bunched region, a proximal end and a distal end attachable to a port of an endoscope, wherein said intermediate bunched region being elastically stretchable upon pulling of one of its ends,
a tubular guide member through which the flexible shaft of the endoscopic tool can be longitudinally displaced when the flexible shaft is being either retracted from a port of an endoscope or protracted thereinto,
wherein the distal end of the first protective portion and the proximal end of the second protective portion being connected to the tubular guide member to bring the first protective sleeve portion in flow communication with the second protective sleeve portion, wherein bunching of the second protective sleeve portion being associated with unfurling of the first protective sleeve portion and with retracting the tool shaft from the port of the endoscope, and wherein elastic stretching of the second protective sleeve being associated with bunching of the first protective sleeve portion and with protracting the tool shaft into the port of the endoscope.

16. The enodscopic system as defined in claim 15, wherein said distal end of said second protective sleeve portion being provided with an adaptor for connecting to the proximal port of the endoscope and said flexible shaft of the endoscopic tool being retractable from the proximal port or protractable thereinto through the protective sleeve.
